(19) United States
(12) Patent Application Publication
(10) Pub. No.: US 2010/0227340 A1
(43) Pub. Date: Sep. 9, 2010

(54) METHOD OF ANALYSIS, DETECTION AND CORRECTION OF FOOD INTOLERANCE IN HUMANS

(75) Inventors: Arkady Rozenshteyn, Beachwood, OH (US); Marina Rozenshteyn, Beachwood, OH (US); Anatoly Volkov, Moscow (RU)

Correspondence Address: RANKIN, HILL & CLARK LLP, 38210 GLENN AVENUE, WILLOUGHBY, OH, 44094-7808, US

(73) Assignee: IMMUNOHEALTH INTERNATIONAL, LLC, Lyndhurst, OH (US)

(21) Appl. No.: 12/376,453

(22) PCT Filed: Sep. 5, 2008
(86) PCT No.: PCT/US08/10399

§ 371 Date: Feb. 5, 2009

Related U.S. Application Data
(60) Provisional application No. 60/971,081, filed on Sep. 10, 2007.

(51) Int. Cl. G01N 33/53; C12Q 1/02

(52) U.S. Cl. 435/7.92; 435/29

(57) ABSTRACT

A method of detection and correction of latent food intolerance (LFI) in humans is described. The method allows, through utilizing a dynamic analysis of a specific test for LFI, assessing the degree of food intolerance and devising an individualized diet that excludes food items which cause latent food intolerance, as well as food items which cause classic allergic reactions. Thus, this method promotes health throughout an individual's life by avoiding and correcting the negative effects of food items that act as immune antagonists.

### CROSS REFERENCE TO RELATED APPLICATIONS

This application is a national stage application of International Application No. PCT/US2008/010399 filed Sep. 5, 2008, which claims priority upon U.S. provisional application Ser. No. 60/971,081 filed Sep. 10, 2007, which is hereby incorporated by reference.

### FIELD OF THE INVENTION

The presently disclosed embodiments are directed to the field of dietetics, and relate to the prevention and treatment of chronic conditions in humans caused by “latent food intolerance” (LFI) or “delayed food allergy”. The term “latent food intolerance” as used herein, refers to a latent anomalous response of the digestive and immune systems to certain food items, consumption of which, particularly over a prolonged period of time, results in various functional disorders in an individual.

### BACKGROUND OF THE INVENTION

At the beginning of the twenty-first century there was a sudden and significant rise of chronic disease among humans throughout the world. Symptoms of digestive disorders occur in approximately 26% of the population of the United States, and 41% of the population in the UK. A variety of disorders are also associated with obesity. Based on current medical data, 45% of all cases of hypertension in the US, 85% of diabetes, and 35% of ischemic heart disease occur in obese individuals. And, the body mass index (BMI) in 30% of the adult population and 50% of the elderly population of the economically developed nations significantly exceeds normal or recommended BMI values, and these numbers continue to rise. Despite significant medical and scientific progress, and enormous resources expended on healthcare in developed nations, increase in disease occurrence has been observed in nearly all technologically advanced countries. This phenomena has been termed, “disease of civilization”. These diseases include, among others, obesity, heart disease, hypertension, arterial disease, diabetes, allergies, etc. It has become evident that the modern pharmacological approach to prevention and treatment of chronic disease must be substantially reevaluated and supplemented. Accordingly, a need exists for at least a partial solution to these problems.

In the last 10 to 15 years, a new understanding relating to the origin of chronic disease has emerged, leading to fundamentally new concepts of prevention and treatment. It is now believed that one's health can be damaged despite the consumption of exclusively high quality, so-called “ecologically pure” foods. Every individual, in response to a regular and prolonged consumption of certain foods traditional to that individual, can develop reactions that, although not classified as classic allergies, are capable of adversely affecting the individual's health. These types of conditions, referred to as “latent food intolerance”, “delayed allergies”, “latent allergies”, and “type 3 allergies” have been difficult to diagnose in recent times, until the emergence of new methods allowing the identification of “immune-antagonistic foods” that cause persistent pathological reactions in a given patient. Elimination of these foods from the diet results in the alleviation of the symptoms, and, frequently, complete cure.

Latent food intolerance or LFI was first described by an American physician Herbert Rinkel back in the 1930s. Although ancient physicians strongly suspected the existence of a link between chronic disease and diet, Herbert Rinkel was the first contemporary physician who confirmed this causal link. Dr. Rinkel discovered that with prolonged consumption of large quantities of certain foods, for example, chicken eggs, a human can develop adverse health conditions. Typically, the affected person does not notice any connection between the food item in question and the symptoms of his or her illness or their exacerbation. Moreover, frequently the person will develop cravings for the food item in question, since the consumption of this food item results in a temporary relief of the symptoms. The food item in question may not cause a characteristic response to detection of allergens, but its pathogens can be detected by other methods. The exclusion of this food item may result in temporary worsening of the condition, somewhat similar to withdrawal symptoms in drug addicts. Shortly following the exclusion of the food item, symptoms of the chronic illness begin to diminish. In the event the patient, during the “withdrawal phase”, resumes consumption of the pathogenic food item, the reaction can be acute.

Regrettably, the “masked food intolerance” syndrome, first described by Dr. Rinkel, did not attract the attention of allergologists, and most practitioners in the field are not familiar with his works. Weak interest in this condition is possibly related to difficult and unreliable methods of detection of the pathogenic food items.

Practitioners specializing in latent food intolerance assert that this condition affects 60 to 80% of the population of developed nations. Despite this, the condition remains largely undiagnosed. This is explained, to a large degree, by the fact that technological methods of detection of foods causing LFI have only emerged in recent years. Prior to these methods, many practitioners disputed the very existence of LFI.

It has long been known that different foods and in different combinations are not digested the same. However, up until the end of the nineteenth century, dietology did not exist as a science, and dietary concerns were addressed by physicians and private chefs. In the twentieth century, contemporaneously with the development of the modern food industry, various concepts and methods of dietetics and nutrition emerged. These were, in essence, the first empirical attempts by society to recommend particular eating habits in response to the emerging phenomena of mass food production and new practices of food consumption. In the last decade many different diets have been conceived, some gaining particular popularity. The most notable diets are as follows.

Shelton's Diet is based on segregation of foods containing proteins and carbohydrates. This diet has few fixed parameters, but is based upon a belief that digestion of carbohydrates and proteins requires entirely different enzymes and chemical conditions. And so, according to Shelton's philosophy, it is prudent to consume carbohydrate rich foods and protein rich foods separately, and without mixing.

The D'Adamo Diet presupposes six different food groups, corresponding to six different blood types. Dr. D'Adamo's theory represents the first attempt to correlate dietary requirements with biological variances. Conceived before availability of laboratory techniques allowing the precise identification of individual antibodies, this theory is based on a hypothesis that there is a partial correlation between blood type and ethnicity (and, accordingly, natural food tolerance) of an individual.

Perhaps the most widely publicized diet is the Atkins Diet. The underlying theme of this diet is the exclusion of foods rich in carbohydrates.

Bregg's Diet is a combination of the “miracle of starvation” with ideologies of veganism and segregation of foods.

During the last several years of the twentieth century, Montiniak's Diet emerged on the market. In 2003 yet another contemporary diet, the South Beach Diet, created by Agatson, gained popularity, among many others. One of the most popular directions in contemporary dietology is “weight watching”. As will be appreciated, numerous variations of these many diets have evolved.

All of the hundreds of well known and not so well known diets share several common principles as follows.

These dietetic systems generally do not contemplate testing an individual prior to the recommendation of a particular diet. That is, these dieting strategies do not employ exact scientific methods of qualifying and quantifying the effects of specific food items on an individual immune system, in order to ascertain the degree of latent food intolerance of the individual.

None of the known diets take into consideration variances in the digestive and immune systems of specific individuals. Instead, these diets attribute certain criteria to foods, based on caloric values and nutritional content, among others, as though these criteria apply universally to all individuals.

None of the known diets take into account individual variances in the formation and excretion of metabolic by-products, as a consequence of processes of digestion and absorption of food.

None of the known diets take into account the dynamics and condition of the immune system of an individual.

None of the known diets are targeted toward increasing natural food tolerance and consequent adaptation of an individual to a particular dietary habitat.

Accordingly, a need exists for a new approach in assessing and formulating a dietary regiment. It would be particularly beneficial to provide a diet which was premised upon qualitative and quantitative methods of analyses of an individual's immune system and which assessed the potential for latent food intolerance. And, if such latent food intolerance was identified, it would also be desirable to identify the relative degree of such intolerance. Moreover, it would be beneficial to be able to specify particular dietary plans formulated to avoid and/or alleviate or at least significantly reduce problems resulting from such intolerance.

Ever since the discovery of latent food intolerance there have been numerous attempts to develop simple but reliable tests for food immune antagonists. In the early 1980s scientists in the United States developed a diagnostic test for food products affecting leukocytes and thrombocytes in the blood. The test was named ALCAT (Antigen Leukocyte Cellular Antibody Test). The results of clinical trials showed ALCAT to be highly effective. In the 1990s a similar test for immune antagonists, named NutronTest, was developed in England. In parallel with ALCAT and the NutronTest, both of which are cellular tests, scientists developed an immunologic test that is based on antibodies circulating in the blood. These antibodies or immunoglobulins are of type IgG. This test is typically referred to as the YORK test. Recently, there was an announcement of successful clinical trials of the YORK test for patients suffering from various chronic diseases. In 1996 another cellular test, designated as the Prime Test, was developed in the United States.

In addition to tests analyzing specific blood parameters, other food intolerance tests have also been developed. These include, among others, saliva test, rectal markers concentration test, and nitrogen oxide rectal test. All of the aforementioned tests enable practitioners to investigate a specific individual's immune or cellular response to various food products. The response is measured as a change in a specific parameter of a body substance being analyzed by mixing it with extracts of specific food items or by introducing these food items into the digestive system. A typical procedure followed by practitioners in devising an individualized dietary plan is as follows. A blood sample or blood serum sample of a patient is mixed with extracts of selected foods. The sample is analyzed, via cellular or immunologic methods, for the response of the patient's immune system to each tested food item. A list of the tested food items, along with the corresponding quantitative immune response, is then compiled. Food items corresponding to a high response level are excluded from the diet. Food items may be rotated at predetermined intervals. As a rule, it is sufficient to exclude food antagonists from the diet for a finite period of time to achieve a significant positive effect.

Although this procedure frequently identifies problem foods, it is rudimentary at best. Accordingly, there is a need for a new strategy and method by which food antagonists can be identified, and treatment methods can be determined for alleviating food intolerance symptoms resulting from ingestion of the identified food antagonists.

### SUMMARY OF THE INVENTION

The difficulties and drawbacks associated with previous methods and techniques are overcome in the present invention methods and products for treating latent food intolerance.

In a first aspect, the present invention provides a method of identifying foods that contribute to latent food intolerance in an individual. The method comprises defining a group of food items representative of foods available to the individual. The method also comprises obtaining corresponding food samples from the group of food items. And, the method comprises obtaining a blood sample from the individual. Immunological response values in the blood sample to the food items are then identified. The response values are arranged in either descending or ascending order. A function based upon the arranged response values is then defined, where the function includes a linear region and a non-linear region. The food items corresponding to the response values in the non-linear region are identified, where such food items are foods that contribute to latent food intolerance in the individual.

In another aspect, the present invention provides a method of identifying foods that contribute to latent food intolerance in an individual and foods to which the individual is tolerant. The method comprises defining a group of food items representative of foods available to the individual. The method also comprises obtaining corresponding food samples from the group of food items. And, the method comprises obtaining a blood sample from the individual. Immunological response values in the blood sample to the food samples are then identified. A frequency response distribution of the response values is defined, whereby the frequency response distribution includes a contiguous region and a non-contiguous region. A first set of food items corresponding to the response values in the contiguous region is identified, where the first set of food items are those foods to which the individual is tolerant. And a second set of food items corresponding to the response values in the non-contiguous region is identified, where the second set of food items are those foods that contribute to latent food intolerance in the individual.

In yet another aspect, the present invention provides a method, performed by use of a computer system, for identifying foods that contribute to latent food intolerance in an individual. The method comprises defining a group of food items representative of foods available to the individual. The method also comprises obtaining immunological response values from the individual for each of the food items in the group. The method additionally comprises identifying a demarcation point based upon the immunological response values that separates the group into a first group of foods that contribute to latent food intolerance and a second group of foods to which the individual is tolerant.

And, in another aspect, the present invention provides a computer program product for use in a computer system. The computer program product is adapted to implement any of the previously noted methods.

As will be realized, the invention is capable of other and different embodiments and its several details are capable of modifications in various respects, all without departing from the invention. Accordingly, the drawings and description are to be regarded as illustrative and not restrictive.

### BRIEF DESCRIPTION OF THE DRAWINGS

FIG. 1 is a prior art food test panel graphically depicting relative antibody concentrations in response to various foods.

FIG. 2 is a flowchart of a first phase of a preferred embodiment method in accordance with the present invention.

FIG. 3 is a flowchart of a second phase of a preferred embodiment method according to the present invention.

FIG. 4 is a flowchart of a third phase of a preferred embodiment method according to the present invention.

FIG. 5 is a flowchart of a fourth phase of a preferred embodiment method according to the present invention.

FIG. 6 is a conventional graphical representation of the results of testing for responses to various foods.

FIGS. 7-9 are graphs of a representative patient's immunological response to various foods in descending order determined in accordance with a preferred method of the present invention.

FIG. 10 is a listing of permissible and impermissible foods for another representative patient derived from a preferred method according to the present invention.

FIG. 11 is an illustration of a representative distribution of food items based upon their relative compatibility derived from a preferred method according to the present invention for the patient referred to in FIG. 10.

FIG. 12 is a response distribution curve illustrating a contiguous normal response region and a discontinuous abnormal response region determined by a preferred method of the present invention.

FIG. 13 is a representative frequency response spectrum determined in accordance with a preferred aspect of the present invention for the patient referred to in FIGS. 7-9.

FIGS. 14-15 illustrate representative frequency response spectrums for patients exhibiting slight to no food intolerance.

FIGS. 16-17 illustrate representative frequency response spectrums for patients exhibiting a minimal degree of food intolerance compared to a patient free from LFI.

FIGS. 18-19 illustrate representative frequency response spectrums for patients exhibiting a moderate degree of food intolerance compared to a patient free from LFI.

FIGS. 20-21 illustrate representative frequency response spectrums for patients exhibiting a high degree of food intolerance compared to a patient free from LFI.

FIG. 22 is a graph of distribution responses for the patient having slight to no food intolerances whose frequency spectrum was presented in FIG. 14.

FIG. 23 is a graph of distribution responses for the patient having a moderate degree of food intolerance whose frequency spectrum was presented in FIG. 19.

FIG. 24 is a graph of distribution responses for the patient having a high degree of food intolerance whose frequency spectrum was presented in FIG. 20.

FIG. 25 is a graph of distribution responses for the patient having a high degree of food intolerance whose frequency spectrum was presented in FIG. 25.

FIG. 26 is a graph illustrating relationships between numbers of food products and corresponding immune response per patient.

### DETAILED DESCRIPTION OF THE PREFERRED EMBODIMENTS

In accordance with the present invention, it is believed that one of the main causes of globalization of latent food intolerance is the inability of the human body to adapt to rapidly developing food processing technologies of the past decades, and associated adverse global changes in dietary trends. It will be appreciated that humans are an integral component of the earth's biosphere. However, as a result of technological progress, humans have essentially become separated from their natural biological habitat. Instead, society has created its own artificial living environment, including artificial dietary trends. As a result of the abundance and enormous variety of foods, including genetically altered foods, in most civilized countries, the human immune system has been subjected to a wide array of adverse effects from these new foods.

Global processes of ethnic mixing have led to a rapid loss of national dietary cultures and digestive adaptations previously established over many centuries. These processes have resulted in the emergence of a new dietary culture, which is standardized and devoid of national heritage, instead based upon modern food processing technologies and practices. Thousands of varieties of foods and their combinations, not previously included in our diet, are currently offered. Market demands, based on the progress of food processing technologies, result in the continuous practice of food producers and distributors to increase the assortment of foods available for consumptions. Highly efficient technologies of food processing, purification, and storage significantly alter the natural structure of many foods, transforming natural foods into highly refined technological products with new properties and chemical compositions. The adaptive mechanisms of the human digestive and immune systems are simply incapable of keeping pace with all of the foregoing adverse factors.

More specifically, in accordance with the present invention, it is believed that the conflict between the human body and food results from the following factors. A person's migration from one ethnic habitat to another, with different dietary trends and traditions typically occurs in a single generation and often in a very short period of time. This does not allow the human body to adapt and transition to the new dietary practice of the new region. An example of this is any of the recent immigration trends occurring around the world, and particularly in the United States. Another factor believed to contribute to this conflict is the introduction of new imported food items, not historically consumed by the given ethnic group. Yet another factor is the consumption of food items prepared via technological processes that disrupt a food's natural composition and structure, thereby imposing new burdens on the digestive and immune systems of people consuming such foods. Frequent and excessive consumption of certain foods specifically intended for hunger suppression are also believed to be a factor contributing to this conflict. Another factor is the ever increasing consumption of certain biologically active additives and synthesized food components. Moreover, still another factor believed to contribute to this conflict is the increasing consumption of genetically altered food products. And, yet another factor believed to contribute to these problems is the unnecessary and excessive use of medications. Accordingly, these factors are believed to be the primary causes for globalization of the latent food intolerance syndrome.

Disruption of one's natural food tolerance may be caused by the suppression of the immune system or prolonged exposure to stress. A special, fairly significant, group of people with decreased food tolerance is represented by certain children of mixed couples from different ethnic backgrounds. Natural food tolerance of such individuals can significantly differ from that of their parents, and is not always consistent with the existing familial dietary traditions. Consequently, such children require particular attention to their diet. Of course, all of the above represent only a small segment of pathological factors that form the foundation of immunological conflicts with food, which should be recognized as a divergence of dietary conditions from the needs and natural capabilities of an individual.

Before describing details of the preferred embodiments of the present invention, it is instructive to present the following information as background.

Antibodies also known as immunoglobulins, are proteins that are found in blood or other bodily fluids of vertebrates, and are used by the immune system to identify and neutralize foreign objects, such as bacteria and viruses. Antibodies are typically made of basic structural units—each with two large heavy chains and two small light chains—to form, for example, monomers with one unit, dimers with two units or pentamers with five units. Antibodies are produced by a kind of white blood cell called a B cell. There are several different types of antibody heavy chain, and several different kinds of antibodies, which are grouped into different isotypes based on which heavy chain they possess. Five different antibody isotypes are known in mammals, which perform different roles, and help direct the appropriate immune response to each different type of foreign object they encounter.

Although the general structure of all antibodies is very similar, a small region at the tip of the protein is extremely variable, allowing millions of antibodies with slightly different tip structures to exist. This region is known as the hypervariable region. Each of these variants can bind to a different target, known as an antigen. This huge diversity of antibodies allows the immune system to recognize an equally wide diversity of antigens. The unique part of the antigen recognized by an antibody is called an epitope. These epitopes bind with their antibody in a highly specific interaction, called induced fit, that allows antibodies to identify and bind only their unique antigen in the midst of the millions of different molecules that make up an organism. Recognition of an antigen by an antibody tags it for attack by other parts of the immune system. Antibodies can also neutralize targets directly by, for example, binding to a part of a pathogen that it needs to cause an infection.

Antibodies exist in different varieties known as isotypes or classes. In placental mammals there are five antibody isotypes known as IgA, IgD, IgE, IgG and IgM. They are each named with an “Ig” prefix that stands for immunoglobulin, another term for antibody, and differ in their biological properties, functional locations and ability to deal with different antigens.

In accordance with one aspect of the present invention, fundamental deficiencies in established methods of detection of food intolerance and dietary optimization have been identified.

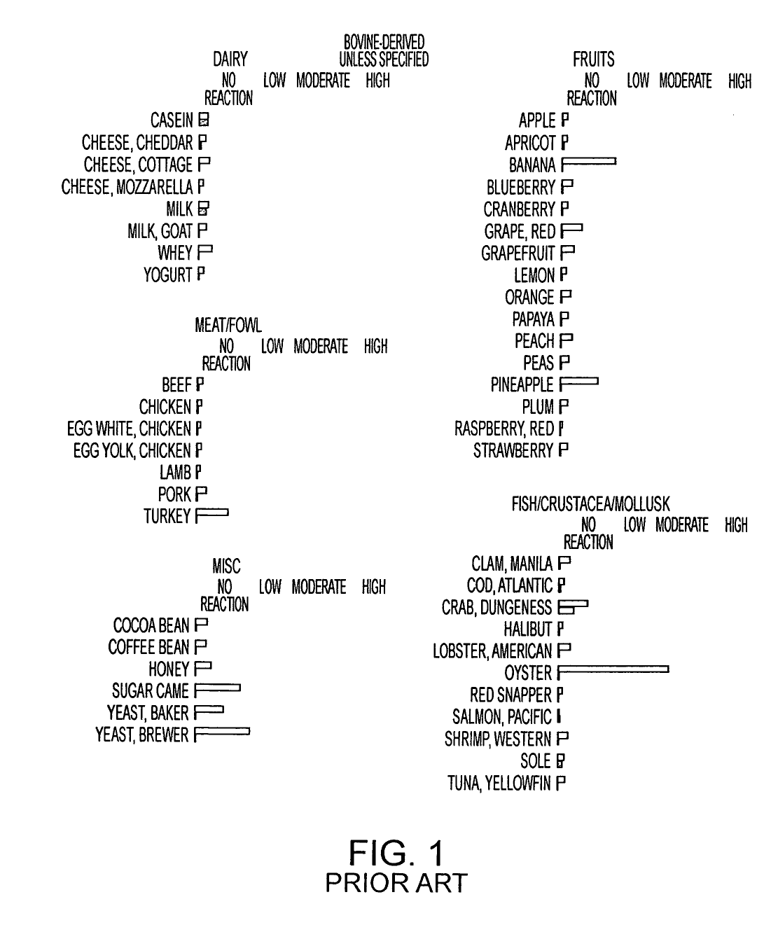

One deficiency relates to the selection of criteria for identifying food antagonists. The terms “food antagonists” or “food immune antagonists” as used herein, refer to foods or agents in foods that have a detrimental effect upon the health of an individual, and which can typically be identified by the individual's immunological response to that food agent. Typically, criteria for identifying food antagonists are derived from the results of known specific tests such as for IgE immunoglobulins in food allergy testing. Such derivation is either subjective, or is done in accordance with principles adopted by allergologists. Generally, these principles suggest that all items that cause an immune response, as measured by the concentration of IgG immunoglobulins in the blood serum, based on traditional allergologist tests for class IgE immunoglobulins are considered allergens or more specifically, food antigens. In the event the measured values of a specific test for an individual do not exceed a predetermined value of the established IgE reference scale, then, according to prevailing methods of interpretation, the individual in question does not have latent food intolerance. For example, FIG. 1 illustrates a conventional food test panel where response values for various foods are graphically illustrated as bars, the lengths of which are proportional to the response values. White bars represent relative IgG concentrations and black bars represent relative IgE concentrations. Each bar extends into one of four quartile regions of the reference scale. These quartile regions are designated as “NO REACTION”, “LOW”, “MODERATE”, and “HIGH.” Thus, according to current conventional methods of interpretation, the individual whose test results are depicted in FIG. 1 does not suffer from latent food intolerance because none of the bars extend into the highest quartile designated as “HIGH”, i.e. exceeding 0.75 of the reference scale. The only food of potential concern according to these test results is oysters, since the IgG bar extends into the “MODERATE” range, i.e. greater than 0.5 of the reference scale.

Historically, results of such tests conducted by a number of American and foreign laboratories are interpreted in accordance with standard allergologic criteria utilized in tests for true (i.e., type 1) allergies. And so, any food items causing a response characterized by the concentration of a test parameter greater than 0.75 of the reference scale are automatically eliminated from the individual's diet.

Since processes taking place in the body are dynamic in nature, a single test of any kind merely represents a statistical sample that does not allow a reliable characterization of the fundamentals of an individual's digestive system. This in turn, precludes a reliable definition of the patient's natural food tolerance and intolerance. At present, there does not exist a method that provides, based on the results of specific tests and identification of food antagonists, complete elimination of excessive strain on the immune system caused by LFI, and which facilitates the adaptation of an individual to their dietary habitat.

The present invention is based upon a new dietary concept and provides new strategies, techniques, methodologies and associated algorithms to analyze, assess, treat and ideally correct a wide array of disorders ultimately stemming from latent food intolerance. The present invention method is based on a sequential detection and elimination strategy of food antagonists from a diet, as well as creation of an individualized plan of dietary rehabilitation that eliminates the effects of food antagonists on the immune system and raises the level of natural food tolerance throughout one's life.

Application of the preferred embodiment techniques, and specifically, a preferred embodiment dietary rehabilitation plan typically result in one or more of the following benefits: normalization of natural metabolic processes, normalization of functions of the digestive system, permanent reduction of excess weight, normalization of processes of extraction of metabolic end products, normalization of blood pressure, lowering of blood cholesterol, elimination of a series of skin conditions, elimination of a series of joint disorders, elimination of chronic fatigue syndrome, elimination of many allergies, increased resistance to infectious and viral disease, decreased dependency on medications, and many others.

In one aspect, the present invention provides a technique for restoring an individual's immune system. The method restores depleted reserves of the immune system, and, at the same time improves overall physical and psychological conditions of the individual. The inclusion of this method into a comprehensive treatment plan intended to combat obesity and associated somatic disease, such as arterial hypertension and metabolic disorders relating to lipid and carbohydrate exchange, serves to significantly increase the efficacy of the specific medical therapy.

Without limiting the present invention, the various preferred embodiment strategies and methods are based on the following concepts.

The digestive function of every individual is based on unique genetic as well as acquired factors. These factors determine the nature and intensity of the individual's immune responses to various food items and their components.

Latent food intolerance is a consequence of either or both, hereditary factors or acquired defects of the digestive function, which manifests in latent immune responses to food items or their components.

Specific responses of the immune system, predicated upon genetically predetermined factors, are generally constant throughout one's life. These responses control unique inherited latent food intolerance of a particular individual to specific food items or their components.

Specific responses of the immune system, conditioned on the acquired factors, are dynamic and change throughout an individual's life.

The results of any single test for latent food intolerance are characterized as a statistical sample, but contain fixed parameters which can be detected by dynamic comparison in a repeated series of tests.

Manifestation and intensity of food intolerance in a specific individual are conditioned upon an assortment of food items and their components to which the individual has latent food intolerance, as well as the relative occurrence of these foods in the individual's regular diet.

Dietary rehabilitation of an individual is a process of detection of food intolerance, followed by the correction of the diet by the exclusion of food antagonists according to the present invention in conjunction with the transition to adherence to general healthy eating habits.

Since blood represents the most important and informative medium in the body, the present invention methods are based upon a view that blood tests are more reliable and representative than tests based on other media. Results in these tests are measured in terms of a quantitative response of the immune system, i.e., quantities of specific antibodies, size and quantity of activated neutrophils, etc. Correct interpretation of the results of a specific test reveals major particularities and problems associated with latent food intolerance in a patient. Results of a test serve as the foundation for a practitioner in devising an individual dietary plan with the purpose of rehabilitation of the immune system and activation of restorative processes in an individual.

The present invention provides various strategies and methods based upon these concepts. In a preferred aspect, the following method or algorithm can be implemented as a computer program preferably comprising the following consecutively conducted preferred phases.

Generally, the methods of the present invention comprise a first phase, i.e. phase I, in which a database of potentially suitable or acceptable food items is defined. This is performed by first identifying a group of food items representative of all food choices available to an individual. Next, a subset from that first group is identified in which the subset includes a lesser number of foods but is still representative of the foods potentially consumed by the individual. Next, a test to identify foods from the subset which evoke an immediate allergic or immunologic response by the individual are identified and eliminated from the subset. The remaining group of potentially acceptable foods is then ready for phase II of the present invention methods.

The second phase, i.e. phase II, involves an analysis of the food items in the remaining group of potentially acceptable foods. The analysis enables the determination of two groups of foods—one group of foods that result, or will likely result, in the occurrence of latent food intolerance in the individual, and another group of foods that do not present such a risk.

The third phase, i.e. phase III, relates to creating a personalized diet for the patient based upon the results of phases I and II. Generally, a database of representative foods is prepared. This may correspond to the previously noted subset of representative foods from phase I. Next, the foods in this database that are within the group of unacceptable foods and those that are within the group of acceptable foods identified in phase II is then determined. The resulting listing of acceptable foods then forms the basis for preparation of a personalized dietary plan.

A fourth phase, i.e. phase IV, is performed after the phases I-III and preferably several months after the patient has begun following the dietary plan as determined from those phases. The fourth phase generally repeats the phases I-III and provides further indication as to foods that may be contributing to any latent food intolerance. The fourth phase may also serve to confirm the dietary plan previously identified and confirm the treatment being administered by the health care professional.

PHASE I—Compilation of Database of Selected Food Items for a Preferred Embodiment Food Intolerance Test

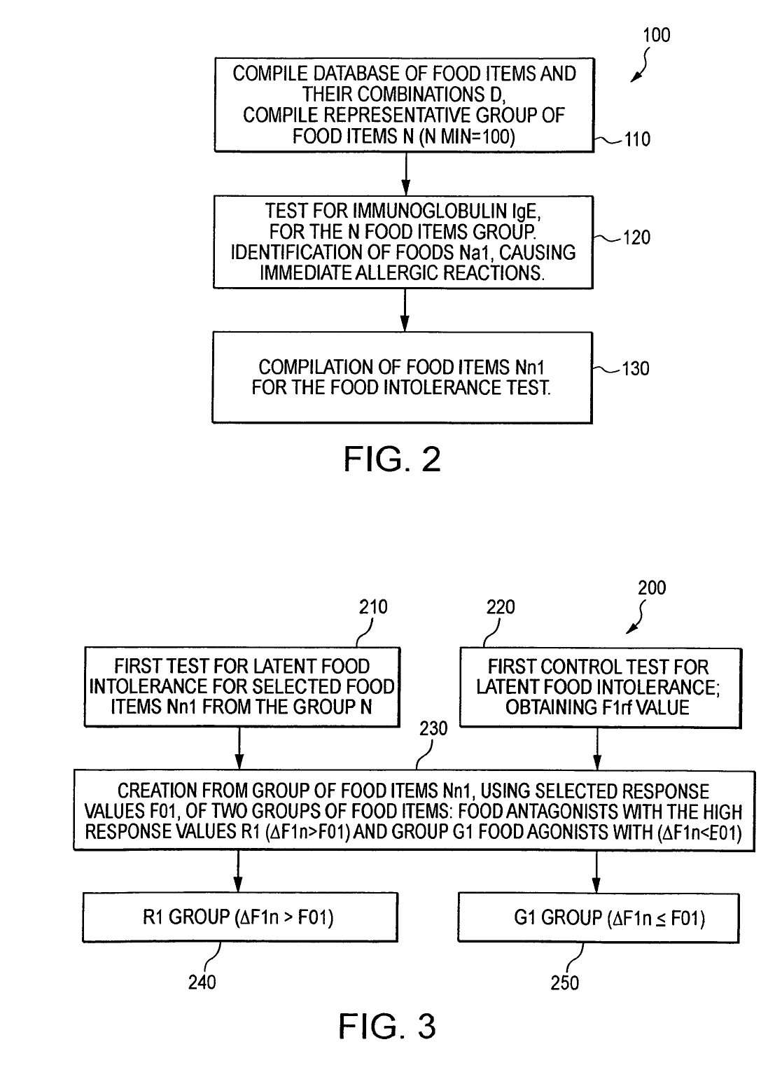

Referring to FIG. 2, in a first preferred phase 100, a database of representative food items is compiled, such as D food items and their combinations (where D is typically less than 1000), reflecting traditional dietary patterns of an individual. A group of representative foods N, where the minimum for N is at least 100, is selected from database D. Extracts or food samples are prepared from each of the selected foods for the test from the group N. These operations are designated as 110 in FIG. 2. It will be appreciated that the present invention also includes D being greater than 1000, and/or N being less than 100.

Blood is drawn from the individual, and tested for immunoglobulins type IgE, responsible for classic allergic reactions of the “immediate type”, for each of the N food items in the group. This step is shown as 120 in FIG. 2.

From the list of food items N, items Na1, those causing immediate allergic reactions, are permanently excluded. The remaining Nn1=N−Na1 food items represent the basis for the following preferred embodiment latent food intolerance test. Compilation of the remaining food items is shown as step 130 in FIG. 2.

### PHASE II—Initial Testing

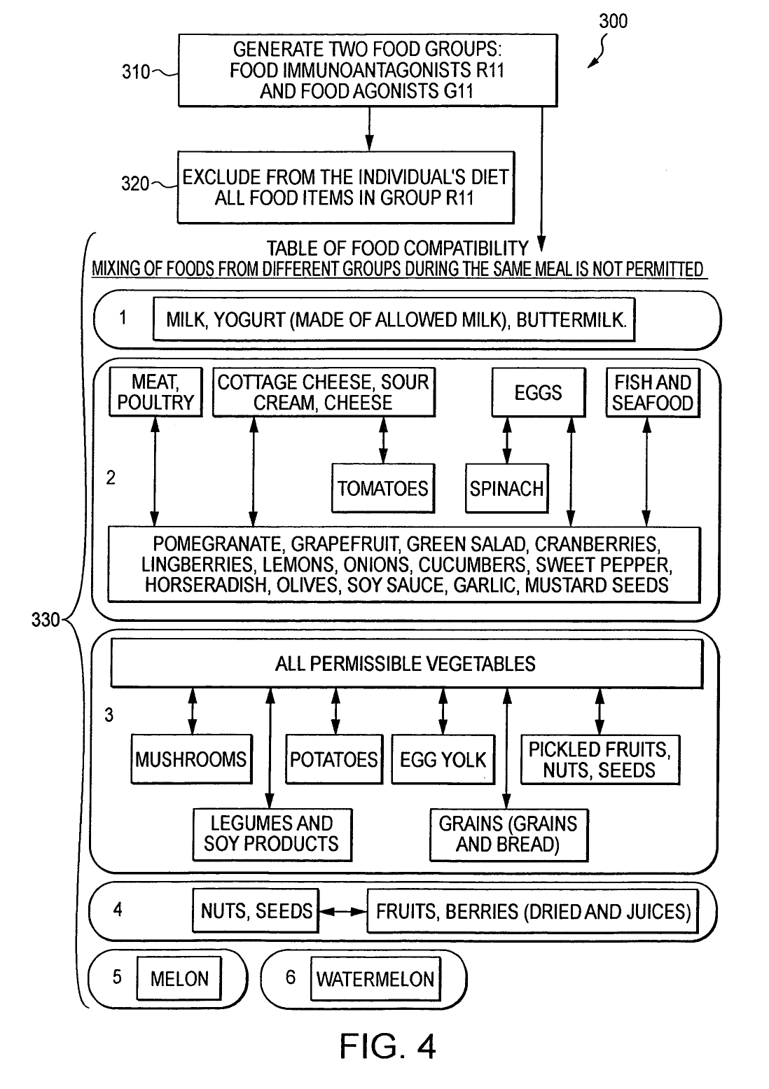

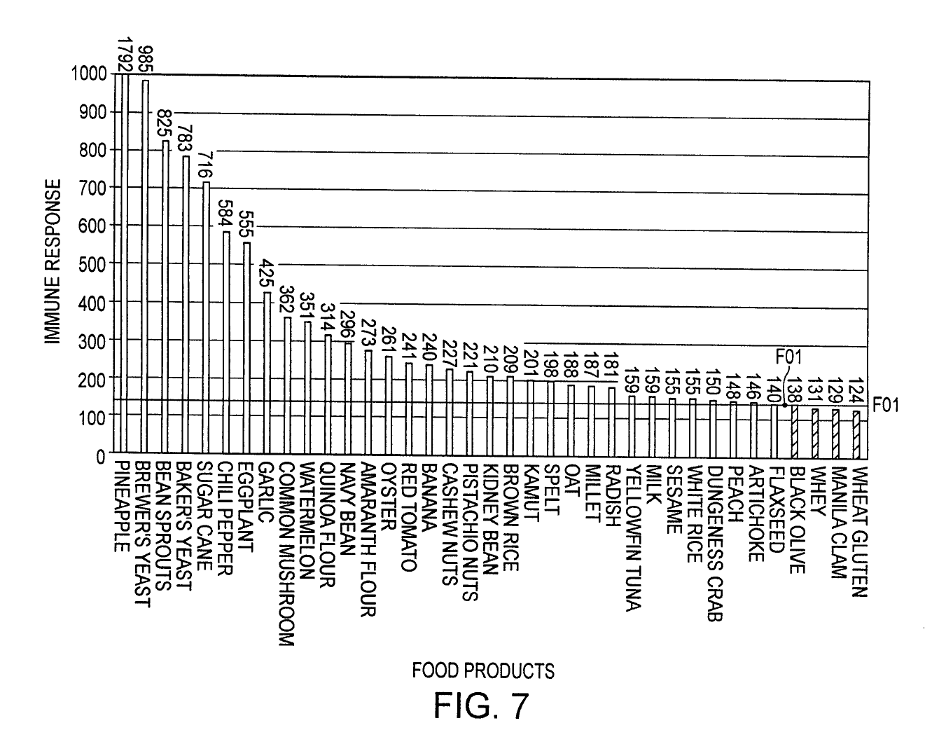

Referring to FIG. 3, in a second preferred phase 200, corresponding blood samples (or blood serum samples) from an individual are mixed, preferably in equal proportions, with the extracts of selected food items from the group Nn1. A first specific test (serum or cellular) for latent food intolerance is performed on selected food items Nn1. This first test may measure resulting concentrations of one or more types of antibodies or immunoglobulins such as IgE and/or IgG in the various samples in response to the respective food items. Measurement of IgG is preferred. This is shown as step 210. The results reflect various degrees of reactivity or response of the body's immune system to each of the food items in the group Nn1, where 1≦n≦N. The results are preferably presented as Fin or functions of response of the immune system in terms of quantitative indicators, characteristic of the specific test being utilized, for each tested food item. Responses can be graphically presented by plotting on the Y axis, concentrations of the antibody such as IgG in blood samples mixed with the extract of a food item, and each n food item from all tested food items in the group Nn1 being identified on the X axis. An example of such a graph of IgG responses is shown in FIG. 6. A commercially available ELISA test can be performed to provide this information.

Enzyme-Linked ImmunoSorbent Assay, also called ELISA ImmunoAssay or EIA, is a biochemical technique used mainly in immunology to detect the presence of an antibody or an antigen in a sample. The ELISA has been used as a diagnostic tool in medicine and plant pathology, as well as a quality control check in various industries. Generally, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light is directed upon the sample, any antigen/antibody complexes will fluoresce so that the amount of antigen in the sample can be measured.

Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

From the blood sample in step 210, K control samples, where 1<K<<N are mixed with k test systems not containing extracts of the food items tested for in step 210. The same test as in step 210 is carried out on the K control samples, producing (F1)k reference functions of response of the immune system for each control sample, and their mean value F1rf is calculated. F1rf represents the mean “noise component” value for the specific test. This is depicted as step 220.

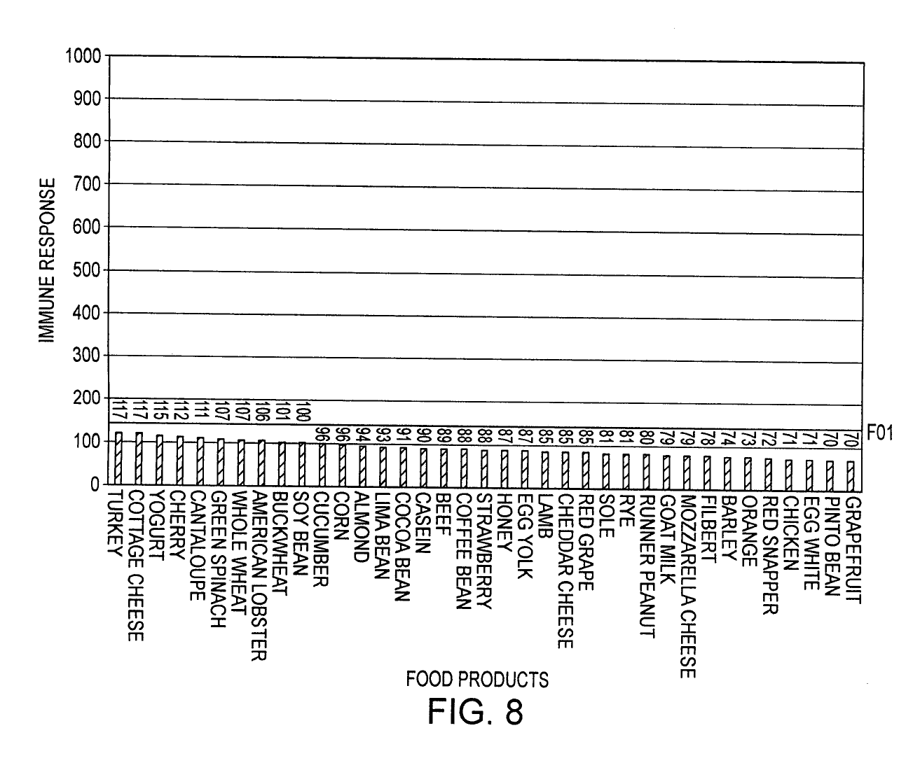

Based on the results of the test in steps 210 and 220, a list of all food items in the group Nn1 is compiled in an order of descending (or ascending) absolute values of the “function of response” where ΔF1n=F1n−F1rf for each n food item. This step is designated as 230 in FIG. 3. FIGS. 7-9 graphically illustrate the individual's immunological response to various foods in descending order. FIGS. 7-9 are representations of responses tn=ΔF1n, where 1<n≦N, in descending order.

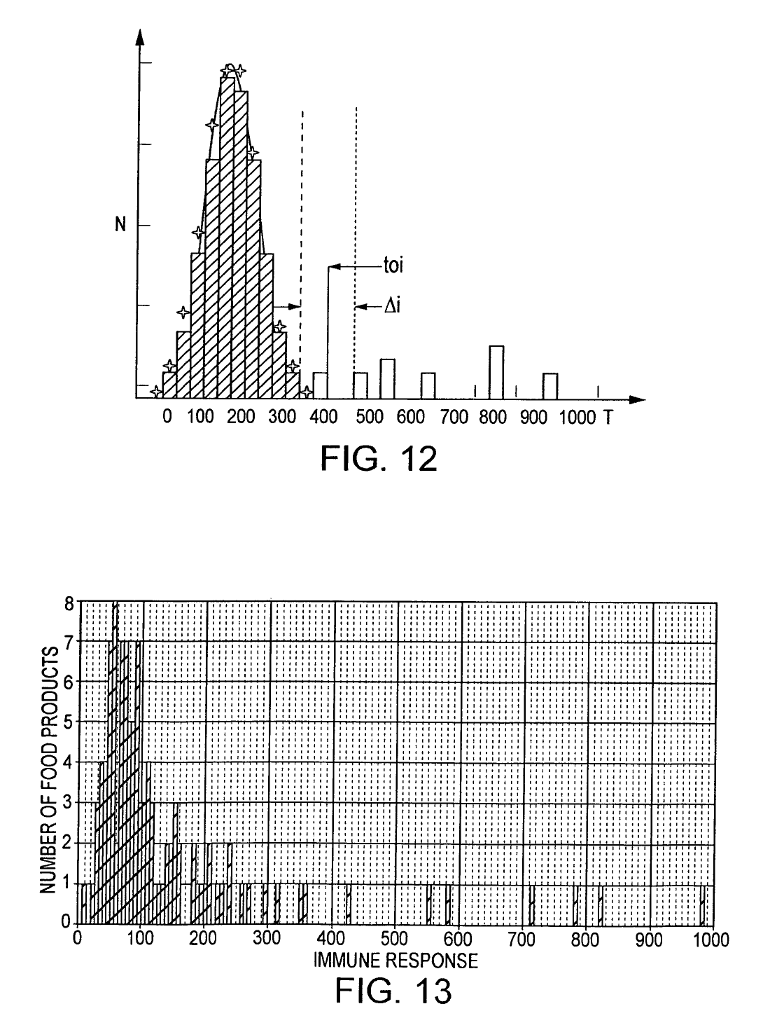

A significant aspect of the preferred embodiment methods of the present invention is identification of a “demarcation point” or separation value F01. Determination of the separation value F01 (in this example F01 is about 139 as depicted in FIGS. 7-9), enables the identification of a group of undesirable food antagonists R1 with response values tn>F01 and a desirable group G1 with response values to ≦F01. Specifically, the demarcation point F01 is preferably the transition point between a linear function and a non-linear function of response values of food items plotted in descending (or ascending) order, such as shown in FIGS. 7-9. The demarcation point F01 also corresponds to the point between contiguous and discontinuous sections of a frequency spectrum of responses, such as shown in FIG. 13, described in greater detail herein. FIGS. 7-9 graphically illustrate descending response values for 111 tested food items.

More specifically, value F01 of the individual's immune response is representative of a criterion of separation of sequence ΔF1=f(n) into two sequences as shown in FIG. 7, thereby generating two groups of food items from the Nn1 list: an R1 group or food antagonists with response values of ΔF1n>F01, and a G1 group, with response values of ΔF1n≦F01, where F1n is the current value of the response level obtained in step 210. The R1 group is shown as 240 and the G1 group is shown as 250 in FIG. 3.

The criterion represented by F01 is selected for each specific blood test, based on the character of the relationship ΔF1n=f(n) for each individual. As noted, the demarcation point may be readily determined by plotting the immune response values for each of the food products tested in either an ascending order or a descending order. Fitting a curve to, or otherwise defining a function corresponding to, the response values enables identification of a non-linear region typically associated with the relatively large response values, and a linear region typically associated with the relatively small response values. The point on the curve or function separating these two regions is the demarcation point. Referring to FIG. 7 for example, the demarcation point F01 is noted. The region to the left of that point is the described non-linear region and the region to the right of that point and extending to FIGS. 8-9, is the linear region. Foods in the non-linear region having immune responses shown as white bars correspond to undesirable foods in group R1. And foods in the linear region having immune responses shown as bars with cross hatching correspond to desirable foods in group G1.

In order to accurately determine the F01 value, in addition to plotting immune responses in ascending or descending order on a graph and then visually identifying the point between linear and non-linear regions, such as generally shown in FIGS. 7-9, statistical analysis of the response values can be performed by mathematical means. The preferred embodiment method also uses statistical analysis of the distribution of food items relative to response values, as depicted in FIG. 13. These aspects are described in greater detail herein.

In the noted preferred embodiment procedure for the second phase, the immunological response to each of the various food items is determined. Although in the description, use of a commercially available ELISA test is noted; it will be appreciated that nearly any test or means of identification can be utilized to obtain immunological response to each of the various food items under review. And, although it is preferred that the test identify the patient's immunological response as indicated by the concentration or changes in the immunoglobulin IgG; the present invention includes the use of other approaches and tests for assessing immune responses.

### PHASE III—First Correction of Food Intolerance

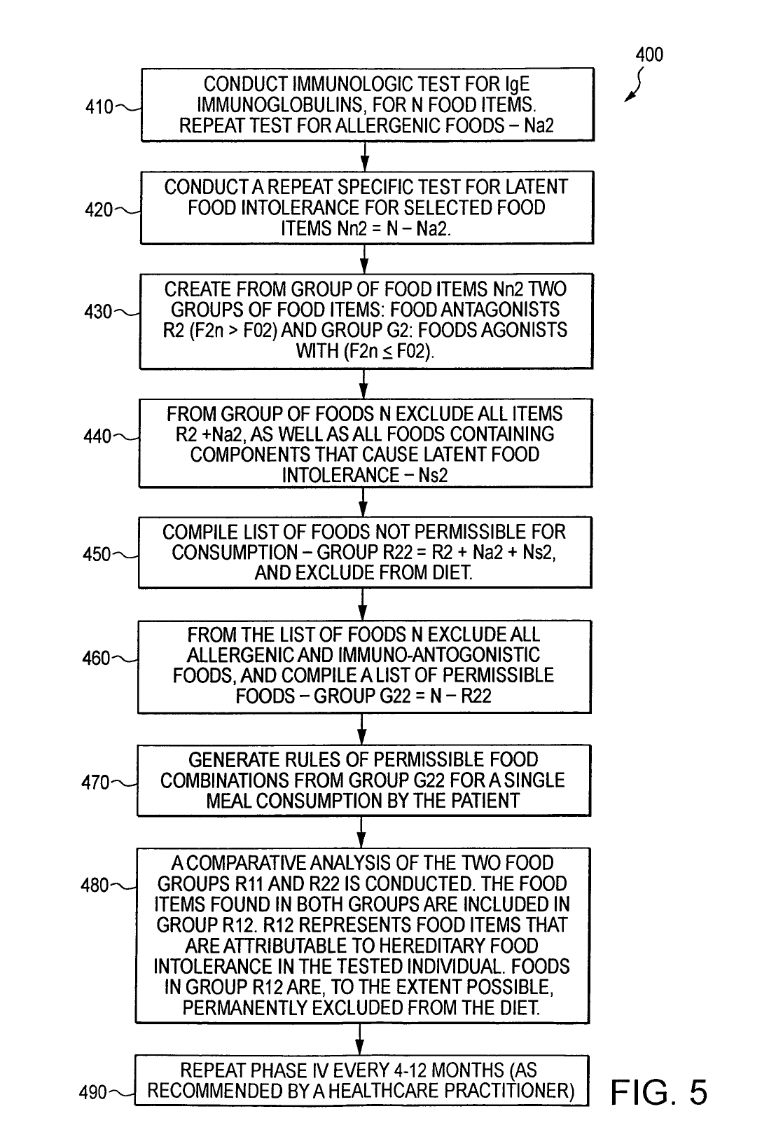

Phase III relates to creating a foundation for an individual's diet based on the results of the previously described tests and analyses. As previously noted, from the total number of food items N, foods causing immediate allergic reactions and their components are identified. This group is designated as Na1. From the total number of food items N, all food antagonists in group R1, with (Fn>F1rf), are excluded as previously described. In the list of food antagonists R1, the main components causing latent food intolerance are determined, and a group Ns1 is defined as foods containing these components. Based on the noted operations collectively designated as 310 in FIG. 4, a list of food items R11=R1+Na1+Ns1 is generated representing foods not permissible for consumption, to be excluded from the diet.

From the list of available food items N all food antagonists discovered in the noted operations are excluded, and a list of permissible food items, group G11 is generated. This is included in step 310 in FIG. 4. Exclusion of food antagonists in group R11 is shown as step 320.

From the group of permissible food items G11 identified in 310 and 320, a set of representative distribution of food items is prepared. This is depicted as 330 in FIG. 4. Preferably the number of categories or groups of foods and their types is determined by the health care professional. However, it is typical to define at least the following groups: a first group of exclusively dairy products; a second group including one or more of meat, poultry, dairy, fish and seafood, vegetables, and fruit; and a third group including other vegetables, dairy, breads and grains, fruits, and various other foods; a fourth group of nuts and seeds, including fruits; and a fifth group of miscellaneous foods. It will be appreciated that the selection of the particular groups and their foods are based upon the expertise of the healthcare professional and the assessment of the needs of the patient. The present invention includes nearly any distribution of food items.

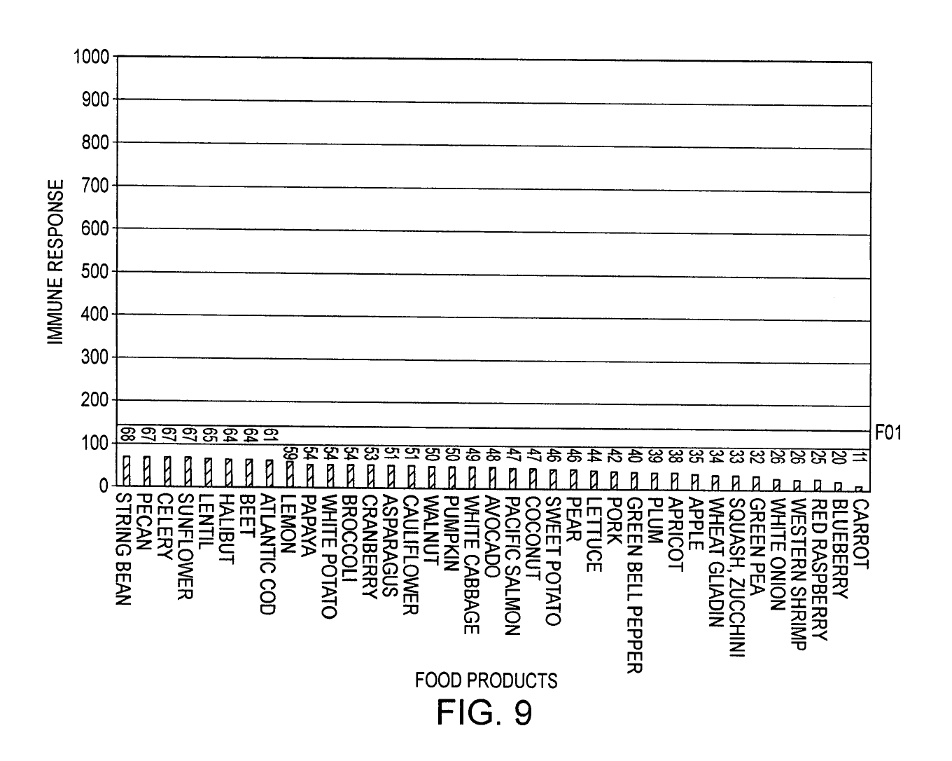

FIG. 10 illustrates a compilation of a representative list of permissible G11 and impermissible R11 foods from the group Nn1, based on the separation value F01 where food immunoantagonists or food antagonists (indicated by a white bar) exhibit high response values, R1 (tn>F01); and food agonists (indicated by a cross hatched bar), group G1 (tn F01) exhibit low response values.

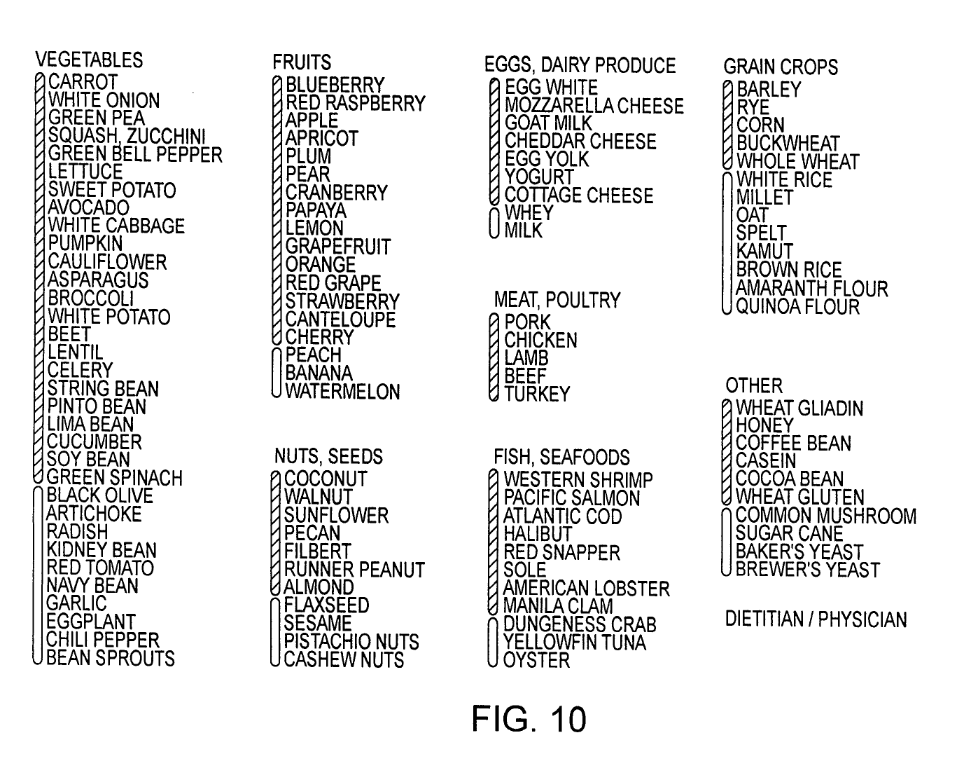

Next, rules of permissible food combinations are generated from group G11 for a single meal consumption by the individual as previously described with reference to 330 in FIG. 4. FIG. 11 illustrates another example of a representative distribution of food items based on their compatibility. FIG. 11 is preferably derived from the patient information depicted in FIG. 10. FIGS. 10 and 11 contain all necessary information for creating a personalized initial elimination diet for a representative patient in accordance with a preferred method of the present invention.

### PHASE IV Second Iteration

In accordance with a preferred aspect of the present invention, a second iteration or second testing, is performed after the initial testing, and preferably, about 12 to 14 weeks thereafter.

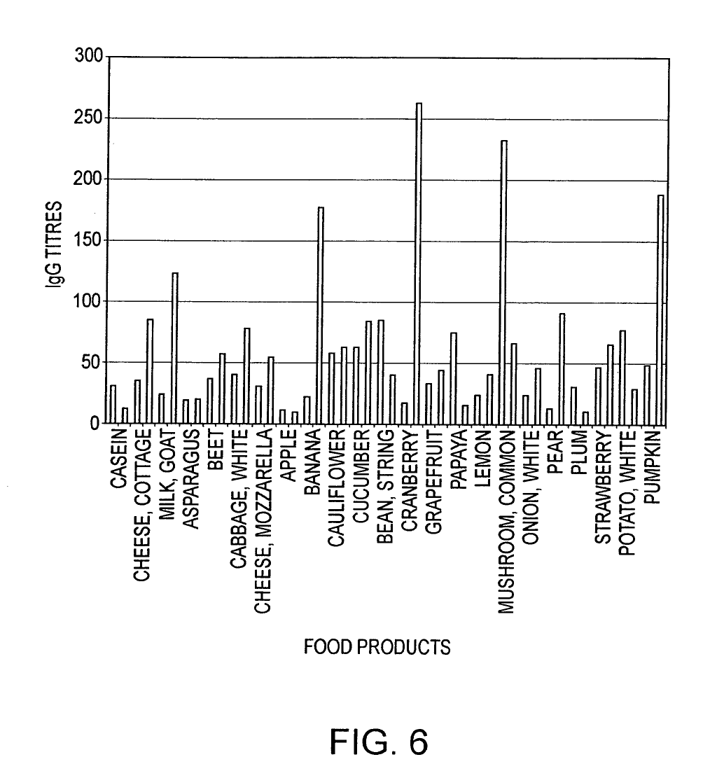

A blood sample is taken from the individual who underwent initial database compilation and testing as described for the first and second phases and initial correction as described for the third phase. A repeat test for type IgE immunoglobulins is performed, as described in the first phase and specifically, step 120 in FIG. 2, to identify classic allergic reactions of type 1 for each of N food items. This operation is shown as step 410 in FIG. 5. The purpose of this procedure is to differentiate type 1 allergic reactions, attributable to the patient's genetic predisposition, from reactions induced by latent food intolerance, which either disappear or are considerably attenuated following the first corrective treatment.

From the list of foods N, Na2 foods are then excluded, which cause type 1 allergic reaction. The remaining Nn2=N−Na2 foods represent the basis of the repeated latent food intolerance test. This operation is included in step 410 in FIG. 5.

The repeat test for LFI is conducted, as per step 210 in the second phase for foods Nn2, and the values of F2n (functions of immune response) are determined. This step is designated as 420 in FIG. 5.

In accordance with step 220 of the second phase, k (1<k<<N) control samples are collected, which are mixed with k test systems, not containing extracts of food products. The specific test is conducted, as per step 210 and the functions of response of the immune system are determined (F2)k for each reference control sample. Their mean value F2rf, is calculated representing the mean value of the noise component for the specific test.

Based on the results, Nn2 foods is compiled in the order of decreasing absolute values of AF2n=F2n−F2rf for each n food item. This information may be presented as shown in previously described FIGS. 7-9.

Demarcation value F02 of the immune response, such as shown in FIG. 7 is introduced as a criterion of separation of sequence ΔF2=f(n) into two sequences, thereby generating two groups of food items from the Nn1 list: R2 group which is food antagonists with response values of ΔF2n>F02, and G2 group with response values of ΔF2n≦F02, where F2n is the current value of the response level during the repeat test. As in the initial test, the F02 value is specific to the tested individual, based on the relationship ΔF2n=f(n) and the essential quantity of nutrients in group G2. This operation is shown as 430 in FIG. 5.

Next, the main components in the list of food antagonists R2 causing latent food intolerance, and food items Ns2 in group N, containing these components are determined. This operation is shown as step 440 in FIG. 5.

In step 450, a list of foods R22=R2+Na2+Ns2 is compiled which represents those foods not permissible for consumption based on the results of the repeat test. These foods are excluded from the diet.

From the list of foods N, all allergenic and immuno-antagonistic foods are excluded. A list of permissible foods, designated as group G22=N−R22 is compiled. This is shown as step 460 in FIG. 5.

Next, rules of permissible food combinations are generated from group G22 for a single meal consumption by the individual as previously explained with regard to operation 330 in the third phase. This is shown as step 470 in FIG. 5.

Based on the results of the consecutive tests, a comparative analysis of the two food groups R11 and R22 is conducted. The food items found in both groups are included in group R12. R12 represents food items that are attributable to persistent food intolerance in the tested individual. Foods in group R12 are, to the extent possible, permanently excluded from the individual's diet. This is shown as operation 480 in FIG. 5.

As needed, the fourth phase is repeated every four to twelve months, or as recommended by a healthcare practitioner. This is shown as step 490 in FIG. 5.

In accordance with another preferred aspect of the present invention, it has been surprisingly discovered that results from conventional IgG immunological response tests can be analyzed in a manner to thereby enable identification of a demarcation point, previously referred to as F01, which then allows ready determination of food antagonists and food agonists. This preferred aspect can be utilized in conducting the procedures of phase II.

FIG. 6 illustrates a conventional graph of IgG ELISA testing results. In accordance with a preferred aspect of the present invention, these results can be analyzed to identify the demarcation point, or graphically presented to readily enable identification of the demarcation point. These aspects are described in greater detail as follows.

In order to determine the type and degree of individual food intolerance, and to find the point of demarcation F01, the present invention also provides a new method of analyzing and/or presentation of test results, utilizing a frequency spectrum of distribution of food items on the scale of response values obtained during the test. In this preferred method, a given scale of 0-T is divided into k equal discreet intervals Δ, where T=K Δ, 1=<K<=T. In order to perform a statistical frequency analysis of each IgG test, the results of the test, seen as individual responses tn in FIG. 6, are presented, preferably via computer program, in the following X-Y coordinates format.

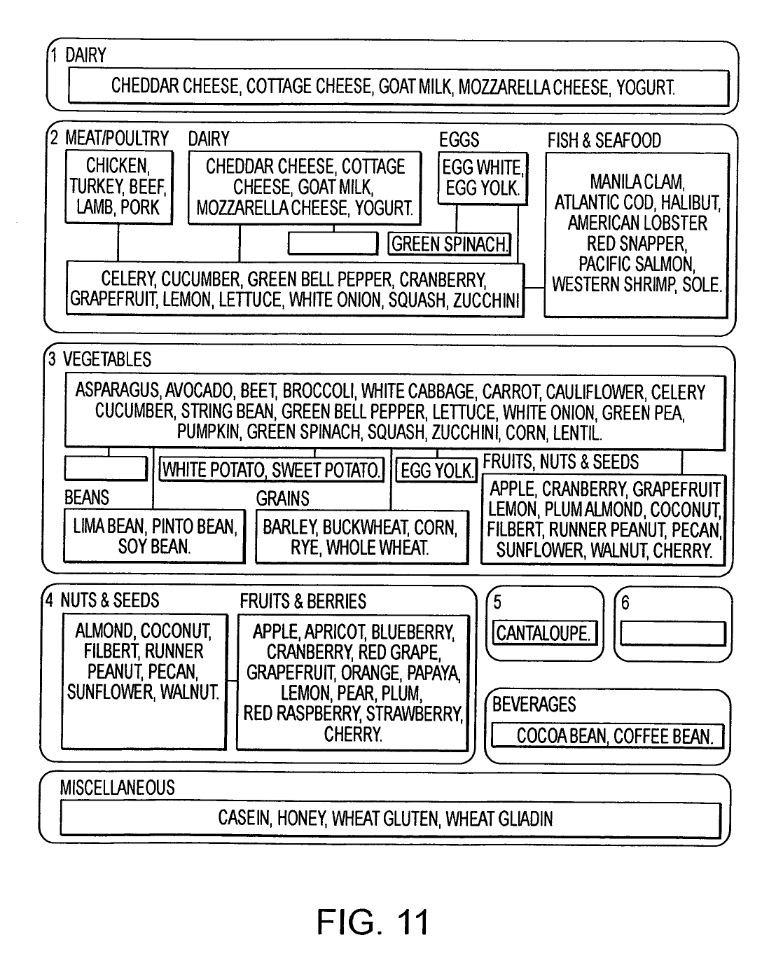

Referring to FIG. 12, for the X axis, a full scale of immune responses 0-T for a given test, is divided into k equal intervals Δi where 1=<i=<k, and i is the sequential number of intervals Δi. The scale of responses tn is in units proportional to the concentration of IgG immunoglobulins in the blood serum sample of the patient for each tested n food item.

For the Y axis, Ni is number of food items with response values t0i±Δt0i/2, in the interval Δi, where t0i is the response value in the middle of interval−Δi. FIG. 12 illustrates values proportional to the probability of occurrence of Ni food item with a response value ti in the interval Δi=t0i±Δt0i/2. FIG. 12 illustrates Ni which is the number of food items with immune response Δti within the interval Δi=t0i±Δt0i/2. In FIG. 12, a relatively contiguous area under the curve defined by the data points (shown as stars) represents a normal distribution curve. The cross hatched bars or contiguous area represents a normal response region. The non-cross hatched bars represent an abnormal response region characterized as a non-contiguous or noncontinuous area.

Actual depiction of a frequency spectrum is shown in previously referenced FIG. 13. On the X axis, the range of response values obtained from the test of interest, such as an IgG ELISA test are presented. On the Y axis, the number of food products that exhibit the particular response value is presented. Thus, for the data presented in FIG. 13, eight food items resulted in response values of about 50. The presentation of the data in FIG. 13 enables identification of the food items within the contiguous region, i.e. appearing as the normal distribution or bell curve generally within the response values of about 0 to about 160. Food items exhibiting response values in the non-contiguous region to the right of the contiguous region, generally constitute the foods that are associated with latent food intolerance.

Such frequency spectrum is unique to each individual being tested for food intolerance such as via an IgG ELISA test. FIG. 13 can be interpreted as a spectrum of immune system responses, represented by IgG immunoglobulins, to Nn1 food items being tested.

Comparison of the function curve in FIGS. 7-9 and the frequency spectrum distribution in FIG. 13 for the same patient allows fine tuning of the demarcation point F01, which is located at the point of transition of the linear region to the non-linear region, as previously described in conjunction with FIG. 7. On the frequency spectrum diagram this point is located where the curve transitions from a contiguous function to noncontinuous or interrupted function, thereby defining the region of food intolerance. In a number of clinical cases this value coincides with the beginning of asymmetry of the right side of the normal distribution curve. Comparing the demarcation point F01 determined from FIGS. 7-9 (about 139), with the demarcation point F01 determined from FIG. 13 (about 160), it will be appreciated that these values are in relatively good agreement with one another.

Based on a comparative study of patient test results, using an IgG ELISA test of blood serum mixed with extracts from 111 food items (the test panel provided by US Biotek, Inc.), the following conclusions have been reached.

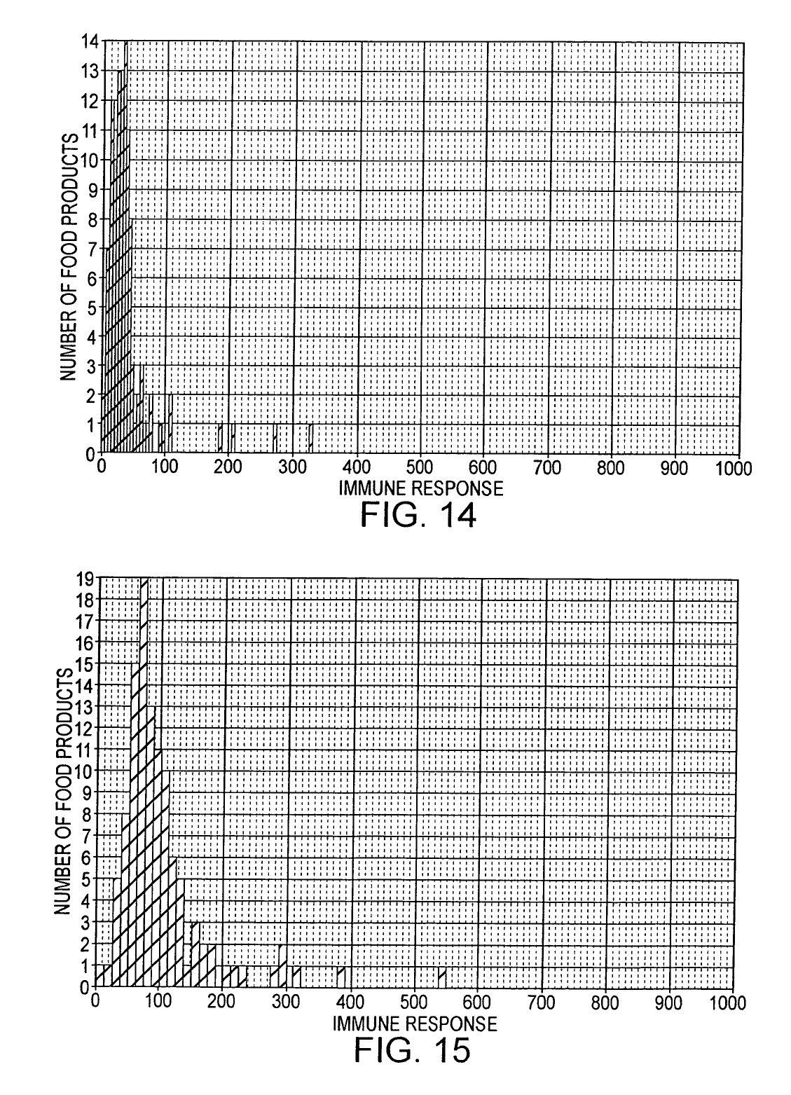

In a case where food intolerance to foods being tested is absent (or only slightly present) in a tested individual, the frequency spectrum of the tested foods on the scale of immune system responses has the appearance of a normal distribution curve, with the standard deviation and mean values being unique to each tested individual. See the frequency spectrums of FIGS. 14 and 15 of two representative patients for example.

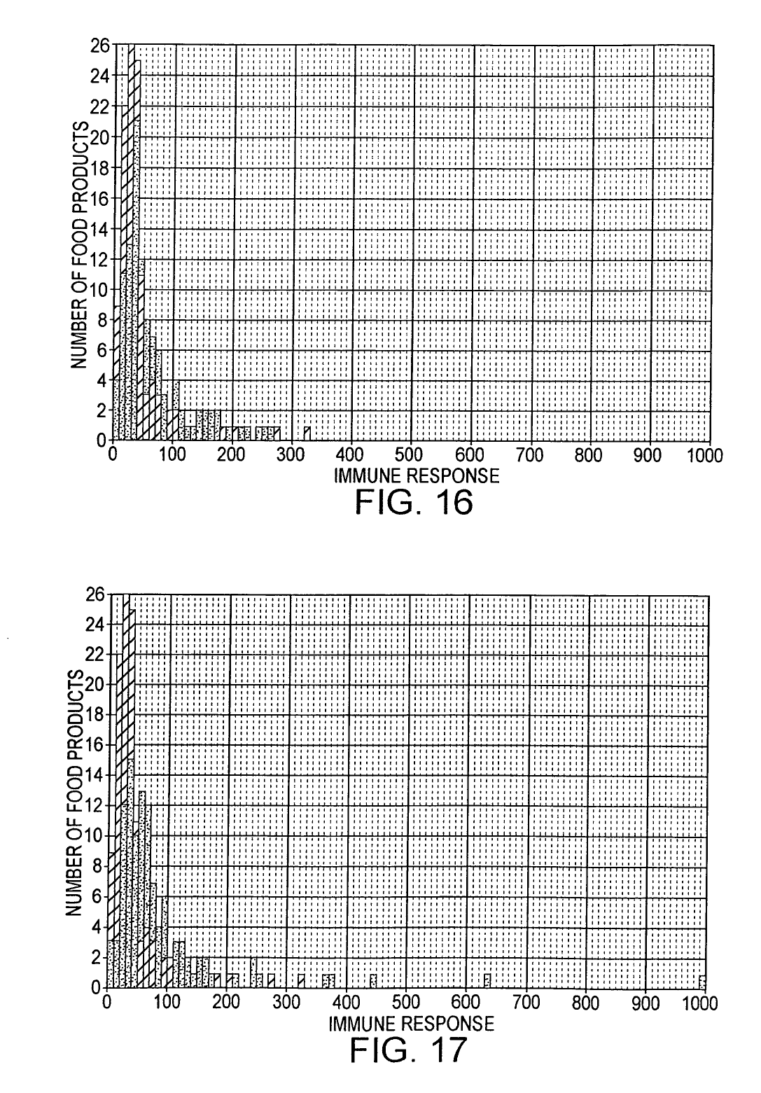

FIGS. 16-21 represent overlapping diagrams of frequency spectrums of patients not affected by food intolerance (consisting of cross-hatched bars) and those affected by varying degrees of food intolerance (consisting of dotted bars). The effect of food intolerance to tested food items is evidenced by asymmetry and distortion of the normal distribution function, characteristic of patients suffering from food intolerance to the tested foods.

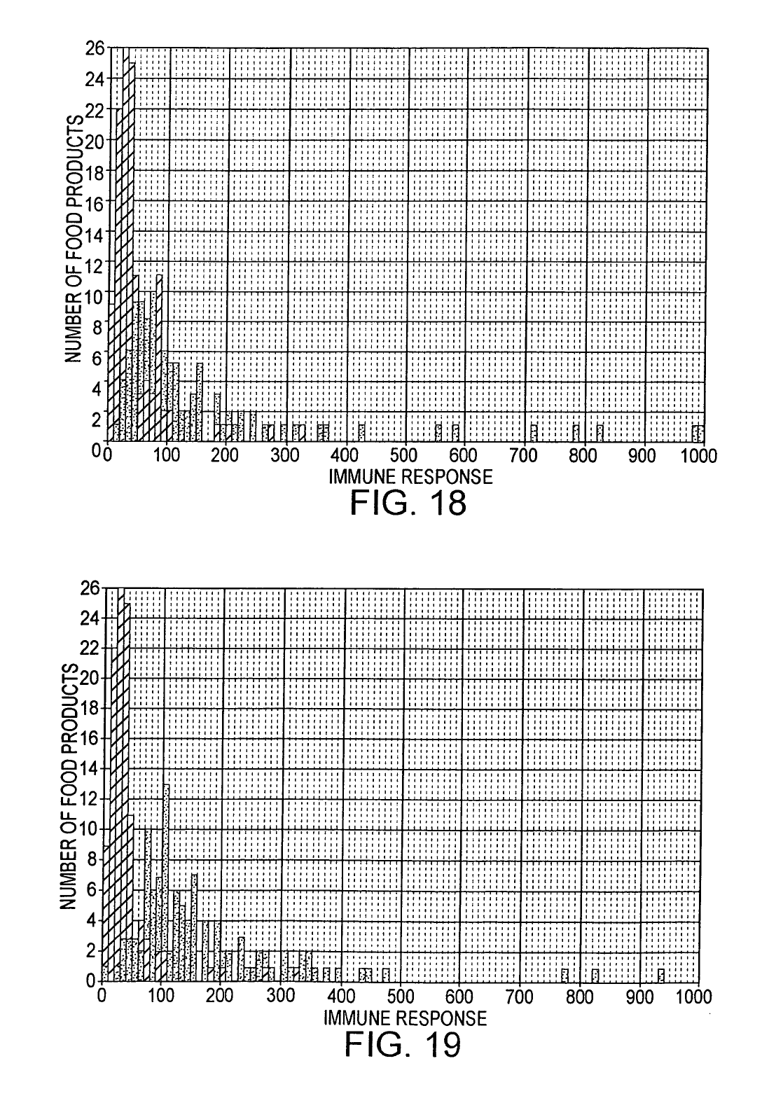

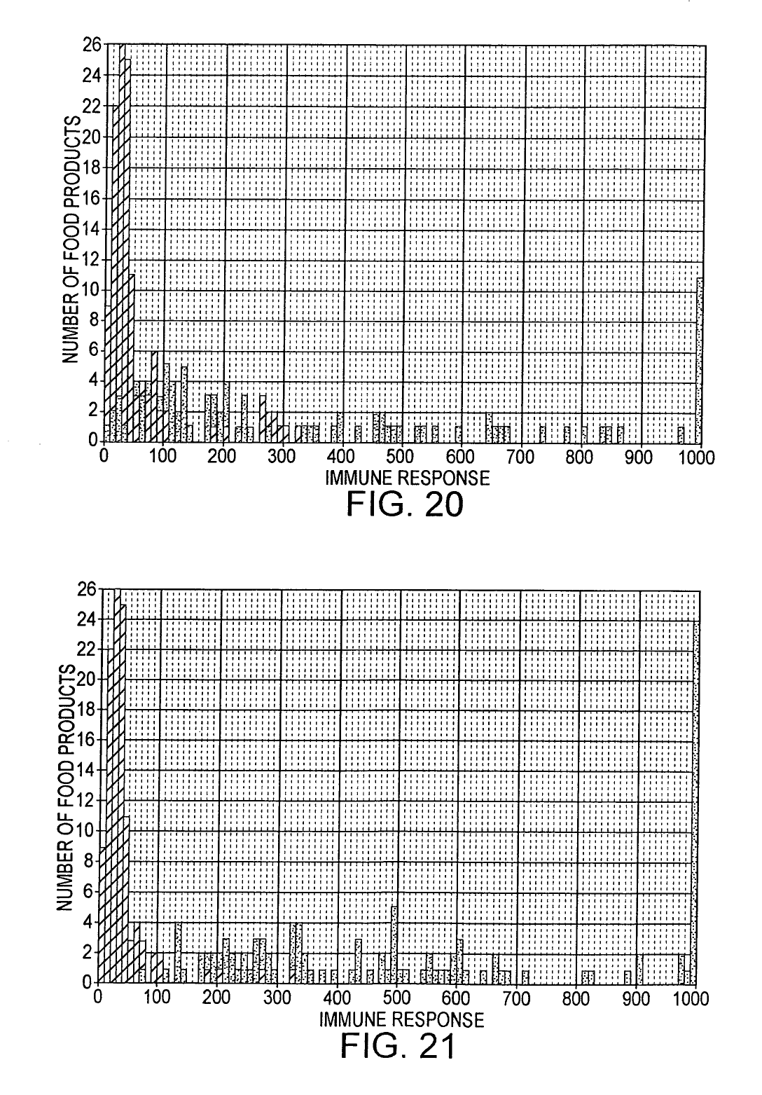

Referring further to FIGS. 14-21, several observations can be made as follows. The frequency spectrum of tested individuals with no food intolerance or only a slight degree of food intolerance, as determined in accordance with the present invention, is depicted in FIGS. 14 and 15. FIGS. 16-17 illustrate frequency spectrums of distribution of tested food items on the scale of immune responses in patients with a minimal degree of food intolerance (dotted bars). FIGS. 18-19 illustrate such frequency spectrums in patients with a moderate degree of food intolerance (dotted bars). FIGS. 20-21 illustrate such frequency spectrums in patients with a high degree of food intolerance (dotted bars). In each of FIGS. 16-21, the patients with some degree of food intolerance (dotted bars) are compared with a patient free of any food intolerance (cross hatched bars).

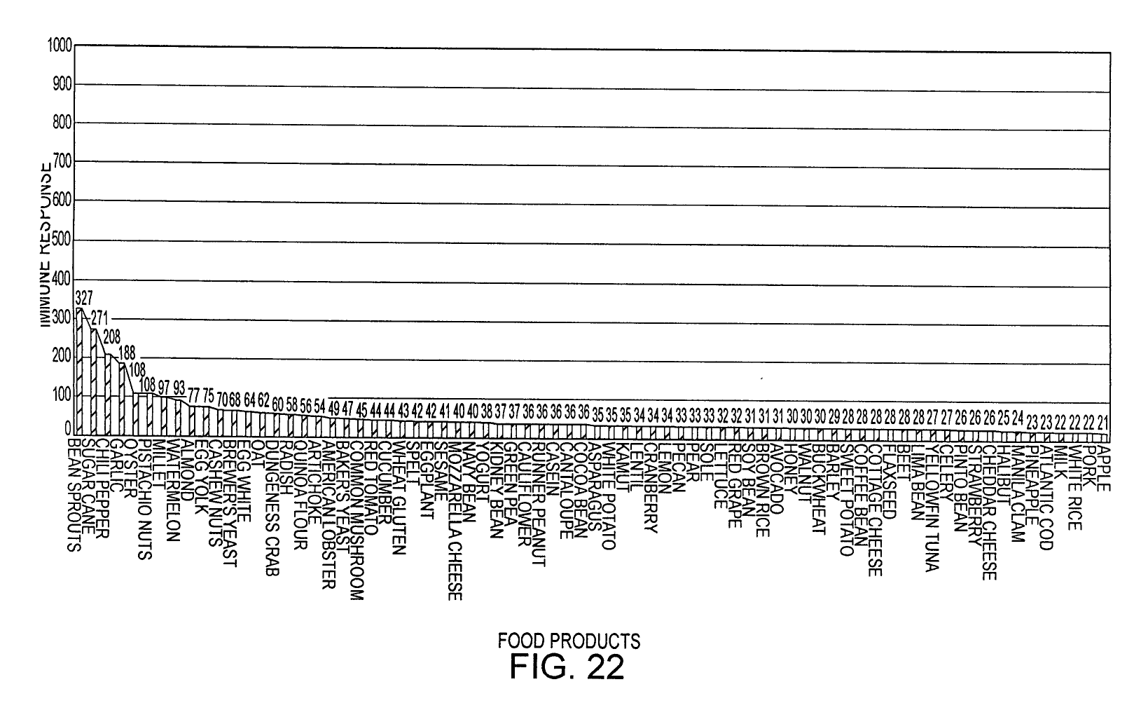

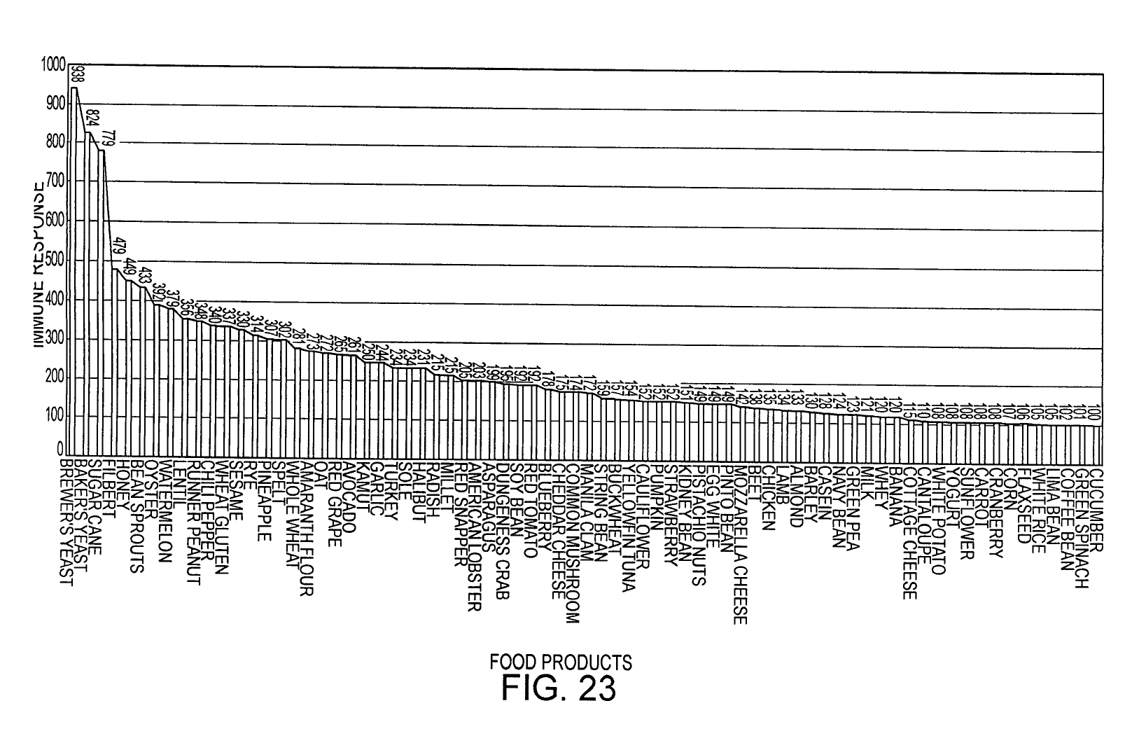

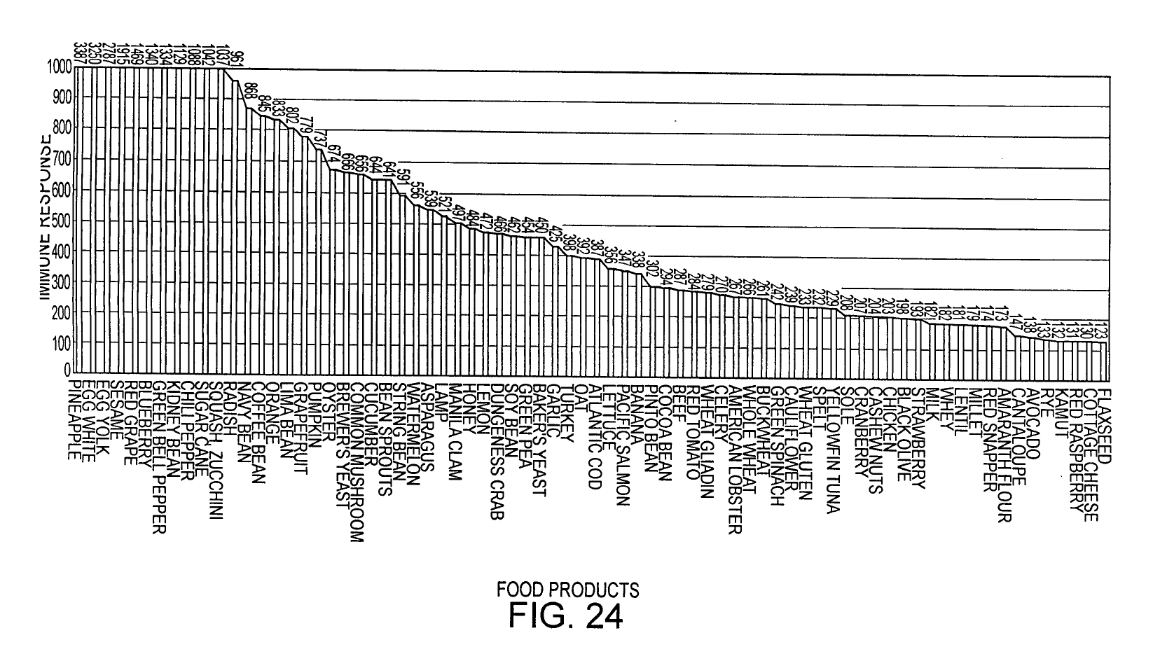

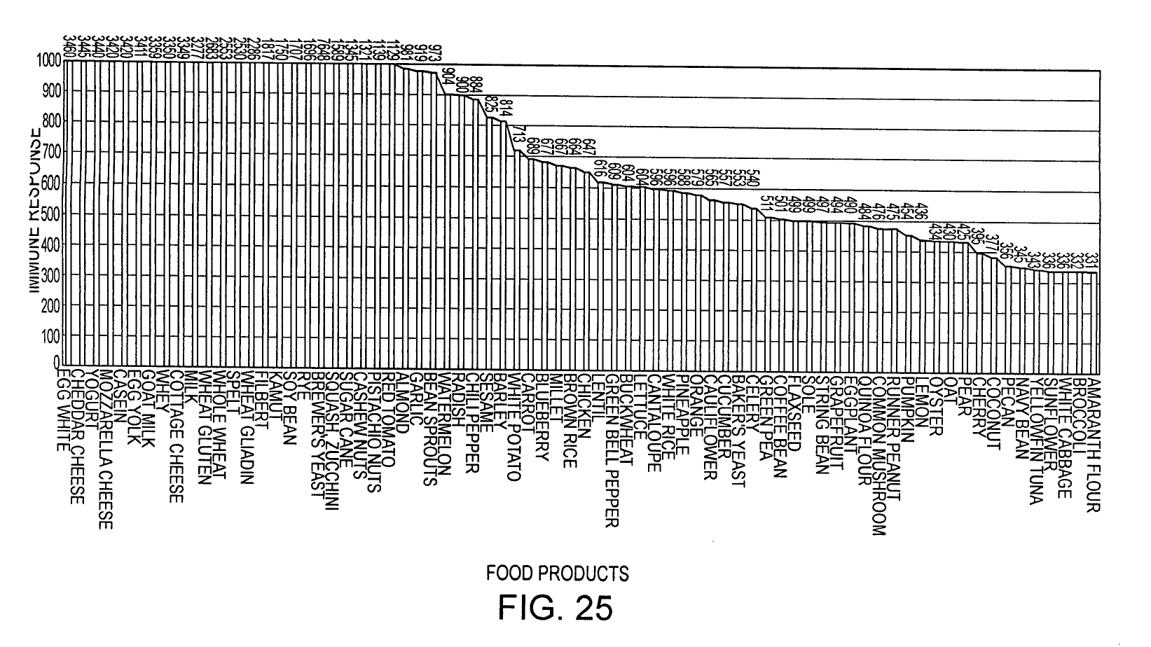

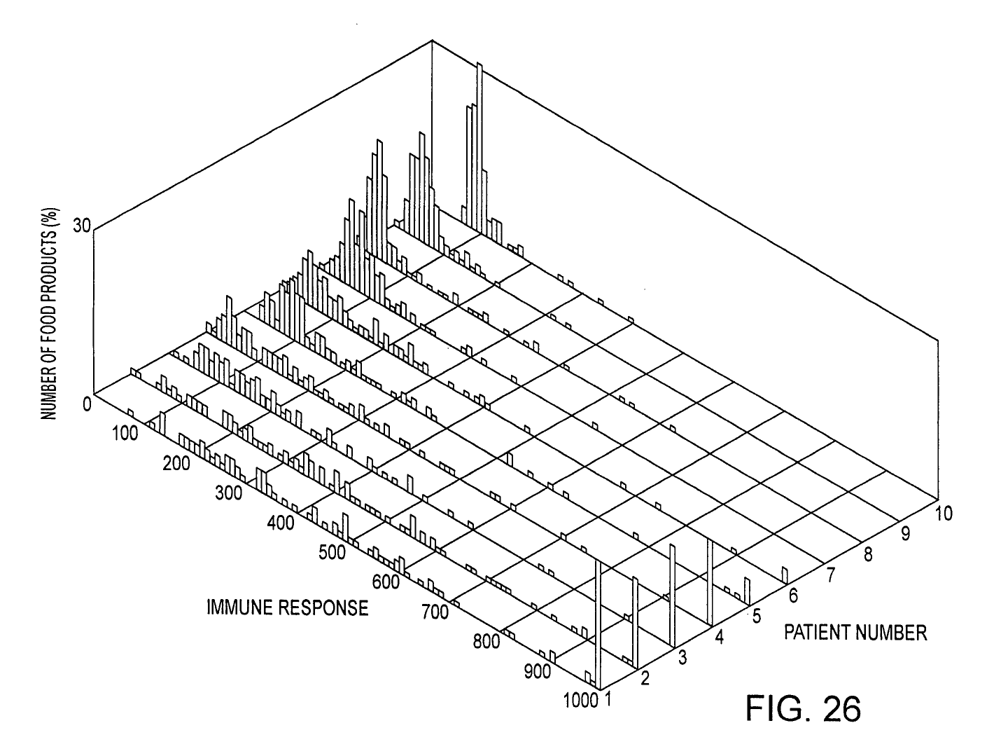

FIGS. 22-25 show distribution of immune responses ti, obtained from testing patients with no detectable or varying degrees of food intolerance to the tested food items. FIG. 22 corresponds to the frequency spectrum in FIG. 14. FIG. 23 corresponds to the frequency spectrum in FIG. 19. FIG. 24 corresponds to the frequency spectrum in FIG. 20. FIG. 25 corresponds to the frequency spectrum in FIG. 21. FIG. 26 is a graph illustrating relationships between numbers of food products and corresponding immune response per patient. Depicted in FIG. 26 is a three-dimensional representation of frequency spectrum distribution of tested food items relative to response of the immune system, characterized by the concentration of IgG immunoglobulins in the blood serum sample, of 10 patients with various degrees of tolerance to the tested food items, from the highest—spectrums 10,9,8 to the lowest—spectrums 1-2. As the degree of tolerance of a patient to the given set of food items is reduced, increased scattering of the normal distribution curve is clearly evident in FIG. 26.

The present invention also provides computer programs, algorithms, or software that conducts a set of instructions to perform one or more of the previously described phases I-IV. In a preferred embodiment, the present invention provides a method, performed by use of a computer or other electronic processing unit, to identify foods that contribute to latent food intolerance in an individual. The method comprises an operation of defining a group of food items representative of foods available to the individual. The method also comprises obtaining immunological response values from the individual for each of the food items in the group. Based upon the obtained response values, a demarcation point is then identified. The demarcation point separates the group of representative food items into a group of unacceptable foods that contribute to latent food intolerance and a group of acceptable foods to which the individual is tolerant.

The present invention also provides computer readable media that comprises programs or algorithms that perform one or more of the previously described phases I-IV; and the previously described preferred method using a computer. Examples of computer-readable media may be any available media, which is accessible by a general purpose computer system. By way of example, and not limitation, such computer-readable media can comprise physical storage media such as RAM, ROM, EPROM, CD-ROM or other optical disk storage, magnetic disk storage or other magnetic storage devices, or any other media which can be used to carry or store desired program code means in the form of computer-executable instructions, computer-readable instructions, or data structures and which may be accessed by a general purpose or special purpose computer system.

In this description, a network or computer network is defined as one or more data links that enable the transport of electronic data between computer systems, processing units and/or modules. When information is transferred or provided over a network or another communications connection (either hardwired, wireless, or a combination of hardwired or wireless) to a computer system, the connection is properly viewed as a computer-readable medium. Thus, any such connection is properly termed a computer-readable medium. Combinations of the above should also be included within the scope of computer-readable media. Computer-executable instructions comprise, for example, instructions and data which cause a general purpose computer system or special purpose computer system to perform a certain function or group of functions. The computer executable instructions may be, for example, binaries, intermediate format instructions such as assembly language, or even source code.

In this description, a computer system is defined as one or more software modules, one or more hardware modules, or combinations thereof, that work together to perform operations on electronic data. For example, the definition of computer system includes the hardware components of a personal computer, as well as software modules, such as the operating system of the personal computer. The physical layout of the modules is not important. A computer system may include one or more computers coupled via a network. Likewise, a computer system may include a single physical device (such as a mobile phone or Personal Digital Assistant “PDA”) where internal modules (such as a memory and processor) work together to perform operations on electronic data.

Those skilled in the art will appreciate that the invention may be practiced in network computing environments with many types of computer system configurations, including, personal computers, laptop computers, hand-held devices, multi-processor systems, microprocessor-based or programmable consumer electronics, network PCs, minicomputers, mainframe computers, mobile telephones, PDAs, pagers, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Utilizing the preferred embodiment methods described herein, significant and beneficial results have been obtained. For example, over 84% of a group of individuals suffering from obesity or a significant overweight condition have achieved a positive long term result, which is exhibited in the form of a significant long term weight loss.

Furthermore, the preferred embodiment methods described herein can serve as a definitive basis for detection and prevention of latent food intolerance, which has been proposed as a significant cause of obesity and related disorders in humans.

It has been demonstrated that frequency spectrums of distribution of tested food items on the scale of immune responses, resulting from conventional ELISA testing for food intolerance based on the level of IgG immunoglobulins in a blood sample, are an informative means of detection and characterization of food intolerance in a specific patient.

Furthermore, examination of the function of distribution of immune responses ti=f(food item) in conjunction with the frequency spectrum Mi=f(ti) for the tested foods allows a definitive determination of the correct point of demarcation between food agonists and food antagonists.

The present invention has wide application. In addition to implementing the various preferred embodiment techniques described herein in therapeutic treatments for patients that are administered by health care practitioners, the present invention can also be provided to individuals as part of a dietary package which the individual may self administer. Thus, it is contemplated that the present invention methodologies could be provided in the form of software programs that an individual could access, either from payment of a one time license fee, or through the use of a continuing license fee. Access to the suite of software would enable the individual to periodically tailor his or her diet, and adjust the current diet to the particular dietary needs of the individual at any particular time.

Moreover, the present invention is not limited to assessing dietary issues of only humans. That is, many different types of animals, and particularly mammals, may benefit from the identification of potential latent food intolerance issues. For example, it is known that race and show horses can develop dietary issues also. It is believed that many of these issues may parallel LFI problems occurring in humans, as described herein. In view of the exorbitant amounts of money that are spent on certain horses, the present invention may find significant application in promoting the dietary health of such animals. Likewise, the present invention may also find application in the fields of prized canines such as show dogs, bulls or breeding cows, or any other animal.

The resulting diet, based upon the proposed methodology, includes a selection of food items for which the frequency spectrum distribution of the tested foods relative to the immune system response values will approach a normal distribution curve.

Many other benefits will no doubt become apparent from future application and development of this technology.

As described hereinabove, the present invention solves many problems associated with previous type devices. However, it will be appreciated that various changes in the details, materials and arrangements of parts, which have been herein described and illustrated in order to explain the nature of the invention, may be made by those skilled in the art without departing from the principle and scope of the invention, as expressed in the appended claims.

What is claimed is:

1. A method of identifying foods that contribute to latent food intolerance in an individual, the method comprising:
defining a group of food items representative of foods available to the individual;
obtaining corresponding food samples from the group of food items;
obtaining a blood sample from the individual;
identifying immunological response values in the blood sample to the food items;
arranging the response values in either descending or ascending order;
defining a function based upon the arranged response values, where the function includes a linear region and a non-linear region; and
identifying the food items corresponding to the response values in the non-linear region, where such food items are foods that contribute to latent food intolerance in the individual.

2. The method of claim 1 wherein identifying immunological response values is performed by mixing blood from the blood sample with a respective food sample in effective portions and measuring such immunological response values.

3. The method of claim 2 wherein identifying immunological response values is performed by conducting an ELISA test.

4. The method of claim 2 wherein identifying immunological response values is based upon assessing changes in the blood sample with respect to immunoglobulin IgG.

5. The method of claim 1 wherein the linear region and the non-linear region are separated by a point of demarcation.

6. The method of claim 1 wherein the group of food items representative of foods available to the individual excludes foods that cause relatively immediate allergic reactions in the individual.

7. The method of claim 1 further comprising:
excluding the food items identified as foods that contribute to latent food intolerance in the individual from the group of food items representative of foods available to the individual, to thereby provide a group of foods acceptable to the individual.

8. The method of claim 1 wherein the food items corresponding to the response values in the linear region represent foods acceptable to the individual.

9. The method of claim 7 further comprising:
defining a dietary plan from the group of foods acceptable to the individual.

10. The method of claim 9 wherein after a period of time during which the individual consumes only foods acceptable to the individual and follows the dietary plan, the method of identifying foods that contribute to latent food intolerance is repeated.

11. The method of claim 10 wherein in the identification of foods that contribute to latent food intolerance is a first set of unacceptable foods and in the repeated method of identifying foods that contribute to latent food intolerance, the repeated identification of foods that contribute to latent food intolerance is a second set of unacceptable foods, the method further comprising:
permanently excluding from the individual's diet, foods that are in both the first set and the second set of unacceptable foods.

12. A method of identifying foods that contribute to latent food intolerance in an individual and foods to which the individual is tolerant, the method comprising:
defining a group of food items representative of foods available to the individual;
obtaining corresponding food samples from the group of food items;
obtaining a blood sample from the individual;
identifying immunological response values in the blood sample to the food samples;
defining a frequency response distribution of the response values, whereby the frequency response distribution includes a contiguous region and a non-contiguous region;
identifying a first set of food items corresponding to the response values in the contiguous region, where the first set of food items are foods to which the individual is tolerant; and
identifying a second set of food items corresponding to the response values in the non-contiguous region, where the second set of food items are foods that contribute to latent food intolerance in the individual.

13. The method of claim 12 wherein identifying immunological response values is performed by mixing blood from the blood sample with a respective food sample in effective portions and measuring such immunological response values.

14. The method of claim 13 wherein identifying immunological response values is performed by conducting an ELISA test.

15. The method of claim 13 wherein identifying immunological response values is based upon assessing changes in the blood sample with respect to immunoglobulin IgG.

16. The method of claim 12 wherein the contiguous region and the non-contiguous region are separated by a point of demarcation.

17. The method of claim 12 further comprising:
defining a dietary plan from the first set of foods.

18. The method of claim 17 wherein after a period of time during which the individual consumes only foods from the first set and follows the dietary plan, the method of identifying foods that contribute to latent food intolerance is repeated.

19. The method of claim 18 wherein in the repeated method of identifying foods that contribute to latent food intolerance, the repeated identification of foods that contribute to latent food intolerance is a third set of unacceptable foods, the method further comprising:
permanently excluding from the individual's diet, foods that are in both the first set and the third set of foods that contribute to latent food intolerances.

20. In a computer system, a method for identifying foods that contribute to latent food intolerance in an individual, the method comprising:
defining a group of food items representative of foods available to the individual;
obtaining immunological response values from the individual for each of the food items in the group;
identifying a demarcation point based upon the immunological response values that separates the group into a first group of foods that contribute to latent food intolerance and a second group of foods to which the individual is tolerant.

21. The method of claim 20 wherein the identifying a demarcation point based upon the immunological response values is performed by:
obtaining a blood sample from the individual:
identifying immunological response values in the blood sample to the food items;
arranging the response values in either descending or ascending order;
defining a function based upon the arranged response values, where the function includes a linear region and a non-linear region, wherein the linear region and non-linear region are separated by the demarcation point.

22. The method of claim 20 wherein the identifying a demarcation point based upon the immunological response values is performed by:
obtaining a blood sample from the individual;
identifying immunological response values in the blood sample to the food samples;
defining a frequency response distribution of the response values, whereby the frequency response distribution includes a contiguous region and a non-contiguous region, wherein the contiguous region and the non-contiguous region are separated by the point of demarcation.

23. The method of claim 20 wherein obtaining immunological response values is performed by mixing blood from a blood sample from the individual with a respective food item in effective portions and measuring such immunological response values.

24. The method of claim 23 wherein obtaining immunological response values is performed by conducting an ELISA test.

25. The method of claim 20 wherein obtaining immunological response values is based upon assessing changes in a blood sample from the individual with respect to immunoglobulin IgG.

26. A computer program product for use in a computer system, the computer program product for implementing a method as recited in claim 20.

27. The computer program product of claim 26, wherein the product includes computer-readable media.
